(19) United States
(12) Patent Application Publication
(10) Pub. No.: US 2015/0119681 A1
(43) Pub. Date: Apr. 30, 2015

(54) METHOD AND APPARATUS FOR SCANNING EXCITATION LIGHT FOR A PHOTOACOUSTIC IMAGE

(71) Applicant: Electronics and Telecommunications Research Institute, Daejeon (KR)

(72) Inventors: Hyun-Woo SONG, Daejeon (KR); Bong-Kyu KIM, Daejeon (KR); Hyung-Wook NOH, Daejeon (KR); Chang-Geun AHN, Daejeon (KR); Eun-Ju JEONG, Daejeon (KR)

(21) Appl. No.: 14/521,321

(22) Filed: Oct. 22, 2014

(30) Foreign Application Priority Data
Oct. 29, 2013 (KR) 10-2013-0129436
Jul. 11, 2014 (KR) 10-2014-0087337

(51) Int. Cl. A61B 5/00; G01N 29/24

(52) U.S. Cl. CPC A61B 5/0095; G01N 29/2418; G01N 29/2462; G01N 2291/023
USPC 600/407

(57) ABSTRACT

Provided is an apparatus for scanning excitation light for a photoacoustic image including a laser delivery device including a rotation reflector configured to receive an excitation laser beam emitted from a laser beam generator, reflect the received excitation laser beam at a certain angle, and radially deliver the reflected excitation laser beam through rotation; a plurality of optical connection lenses disposed on a circumference having a predetermined radius from the rotation reflector and configured to sequentially receive the excitation laser beam while the rotation reflector rotates; and a plurality of optical fiber strands connected with the plurality of optical connection lenses and configured to guide laser light received by the optical connection lenses to a photoacoustic probe.

### CROSS-REFERENCE TO RELATED APPLICATION

This application claims priority to and the benefit of Korean Patent Application No. 2013-0129436 (filed on Oct. 29, 2013) & No. 2014-0087337 (field on Jul. 11, 2014) the disclosure of which is incorporated herein by reference in its entirety.

### BACKGROUND

1. Field of the Invention

The present invention relates to a method and apparatus for scanning excitation light for a photoacoustic image, which may be used to obtain a photoacoustic tomographic image of a biological tissue.

2. Discussion of Related Art

The present invention discloses a method for scanning excitation light for a photoacoustic image, which is associated with a technique for radiating short pulse light to a targeted biological tissue and collecting ultrasonic waves generated in response to the radiated light to acquire a tomographic image of the biological tissue.

Tomography may be performed by excitation laser light having a pulse width of several nanoseconds and a pulse energy of several millijoules to the biological tissue, detecting ultrasonic signals generated at the biological tissue using an ultrasonic wave sensor, and analyzing the signals.

Photoacoustic tomographic images require a technology related to an excitation laser light source that may provide short pulses having high pulse energy. Light is widely radiated using the light source onto a targeted biological tissue desired to be imaged. Thus, an ultrasonic signal may be detected from a region around the targeted biological tissue.

In other words, in order to obtain photoacoustic tomographic images, the technology related to an excitation laser light source that may provide short pulses having high pulse energy is required. In general, the excitation laser light source is very expensive and large.

Document 1 discloses a technology related to a photoacoustic breast scanner that includes a multi-ultrasonic wave measuring device (for example, a transducer) that may be placed in contact with a surface of a biological tissue and then moved for scanning when acquiring a biological image including an absorption characteristic of optical waves in the biological tissue, thus facilitating acquisition of a tomographic image of a biological tissue.

Further, document 2 discloses a technology related to a scanner having an optical fiber that is tightly fixed to a conductor loop vertically to a length direction of the optical fiber and having an end part that can act as a cantilever to be moved. There is disclosed in document 2 a technology related to an optical fiber lateral scanner for a miniature optical fiber probe, in which the optical fiber scanner includes the conductor loop that acts as a permanent magnet and an electromagnet and controls the movement of the end of the optical fiber through modulation of an electric current applied to the conductor loop.

As a conventional technology, document 3 discloses a method of detecting generated ultrasonic signals by bringing a Fabry-Perot interference film sensor head into contact with an object desired to be imaged, exciting the object with laser having pulses of nanoseconds, and sensing an ultrasonic wave, which is generated at the object, because the ultrasonic wave gives modulation to the space layer of the interference film sensor head. The displacement in the space layer varying with time, that is, an ultrasonic wave may be optically detected. There is disclosed in document 3 a technology related to three dimensional noninvasive imaging of the vasculature in the mouse brain using a high resolution photoacoustic scanner, in which a distribution of an ultrasonic wave giving modulation to the space layer and a tomographic image may be obtained by focusing and scanning probe laser with a wavelength having a greatest modulation slope of transmittance to the Fabry-Perot interference film sensor head and measuring modulation caused by reflected light with time.

Existing technologies may detect an ultrasonic signal by widely radiating light to a targeted biological tissue to be imaged. The light source is required to have very high optical pulse energy. In addition, the existing technologies may detect an ultrasonic signal by dividing the light source into several sub-light sources and irradiating certain regions next to the targeted biological tissue with the divided sub-light sources.

The existing technologies use the light source having high optical pulse energy or a plurality of divided light sources to irradiate a wide portion in order to obtain a photoacoustic tomographic image. Thus a technology related to an excitation laser light source of high energy having multiple times an optical output is needed to divide the light source into the plurality of light sources. In general, such a high-energy laser pulse apparatus has a great size and a high price.

Moreover, when energy for each pulse is increased, repetition rate may be reduced and a frame rate (fps) of a photoacoustic tomographic image may be decreased, thereby making the image difficult to obtain in real time.

### PRIOR ART DOCUMENTS

### Patent Document

Patent document 1 (also referred to as document 1): U.S. Patent Publication No. 2002-0035327A1 entitled “Photoacoustic breast scanner”
Patent document 2 (also referred to as document 2): U.S. Patent Publication No. 2006-0285791A1 entitled “Optical fiber lateral scanner for a miniature optical fiber probe”

### Non-Patent Document

Non-Patent document 1 (also referred to as document 3): Laufer, J, et al. (Univ. College London), “Three dimensional noninvasive imaging of the vasculature in the mouse brain using a high resolution photoacoustic scanner,” Applied Optics, Vol. 4 (10), D299 D306, April 2009.

### SUMMARY OF THE INVENTION

The present invention is directed to providing an excitation light scanning apparatus by sequentially radiating an area to be measured with a plurality of sequentially delayed laser light pulses that have a same peak value of a power output as a laser beam generated by a laser generator.

The present invention is also directed to providing an excitation light scanning method and apparatus that may perform high speed scanning using a laser apparatus having relatively low pulse energy for delaying excitation laser light by using a rotation reflector, radiating the delayed laser light pulses onto a plurality of certain measurement areas, and collecting ultrasonic signals received in response thereto to obtain a image.

According to an aspect of the present invention, there is provided an apparatus for scanning excitation light for a photoacoustic image, which includes a laser delivery device, the laser delivery device comprising:

a rotation reflector configured to receive an excitation laser beam emitted from a laser beam generator, reflect the received excitation laser beam at a certain angle, and deliver laser light pulses separated radially through rotation toward a circumference of rotation radius;

a plurality of optical connection lenses disposed on a circumference having a predetermined radius from the rotation reflector and receiving the laser light pulses delivered by the rotation reflector sequentially at an interval; and

a plurality of optical fiber strands connected with the plurality of optical connection lenses, respectively, and configured to guide the laser light pulses incident at the optical connection lenses to a photoacoustic probe.

An apparatus for scanning excitation light for a photoacoustic image, which includes the photoacoustic probe connected with the laser delivery device, and wherein the photoacoustic probe is configured to perform transmitting the laser light pulses and receive an ultrasonic wave generated inside a skin in response to the laser light pulses on a same surface

The photoacoustic probe may include: at least two optical fiber matrix connection modules having the plurality of optical fiber strands collectively connected thereto; an excitation laser output surface having a plurality of laser output holes, the plurality of laser output holes being connected with the plurality of optical fiber strands coupled through the optical fiber matrix connection modules and configured to output the laser light; and an ultrasonic wave sensor matrix module disposed adjacent to the laser output surface and configured to receive an ultrasonic wave generated at targeted tissue due to the laser light radiated to the targeted tissue.

The rotation reflector may be coupled with a driving device rotating at a high speed, and wherein the laser light pulses are input to the plurality of optical connection lenses while the rotation reflector is rotated.

The rotation reflector may be disposed at an angle of 45 degrees or 135 degrees with respect to the excitation laser beam to refract the excitation laser beam by 90 degrees.

The excitation laser output surface may be formed at top and bottom sides of one end surface of the photoacoustic probe, the ultrasonic wave sensor matrix may be formed at a middle side of the end surface of the photoacoustic probe, and the optical fiber matrix connection modules may be formed on top and bottom surfaces of the photoacoustic probe.

The ultrasonic wave sensor matrix module may further include an ultrasonic wave absorber structure configured to prevent an input ultrasonic signal from being reflected.

The ultrasonic wave sensor matrix module may further include an ultrasonic wave focusing acoustic lens to effectively detect an ultrasonic wave at a certain predetermined depth of an examined object.

According to another aspect of the present invention, there is provided a method of scanning excitation light for a photoacoustic image, the method including:

generating an excitation laser beam from a laser beam generator;

reflecting the generated excitation laser beam at a predetermined angle by a rotation reflector and sequentially delivering laser light pulses toward a circumference of a rotation radius;

inputting the laser light pulses to a plurality of optical connection lenses, the plurality of optical connection lenses being disposed in a circular form having a predetermined radius from the rotation reflector;

guiding the laser light pulses input to the plurality of optical connection lenses to a photoacoustic probe through a plurality of optical fiber strands connected to the plurality of optical connection lenses, respectively;

radiating the laser light pulses guided to the photoacoustic probe onto a targeted tissue through a plurality of laser output holes; and

receiving an ultrasonic wave generated at the targeted tissue by the radiated laser light pulses using an ultrasonic wave sensor matrix of the photoacoustic probe.

### BRIEF DESCRIPTION OF THE DRAWINGS

The above and other objects, features, and advantages of the present invention will become more apparent to those of ordinary skill in the art by describing in detail exemplary embodiments thereof with reference to the accompanying drawings, in which:

FIG. 1 shows a structure of an integrated photoacoustic probe that receives a laser photoexcitation and an ultrasonic wave for a photoacoustic image according to an embodiment of the present invention;

FIG. 2 shows a structure of a laser delivery device that outputs an excitation laser beam according to an embodiment of the present invention;

FIG. 3 is a view for illustrating a traveling path of a laser reflected by a rotation reflector 202 of a laser scanning apparatus according to an embodiment of the present invention; and

FIG. 4 shows waveforms of a plurality of laser light pulses output by a laser delivery device according to an embodiment of the present invention.

### DETAILED DESCRIPTION OF EXEMPLARY EMBODIMENTS

Since the present invention may have diverse modified embodiments, preferred embodiments are illustrated in the drawings and are described in the detailed description of the invention. However, it should be understood that the particular embodiments are not intended to limit the present disclosure to specific forms, but rather the present disclosure is meant to cover all modification, similarities, and alternatives which are included in the spirit and scope of the present disclosure. Moreover, detailed descriptions related to well-known functions or configurations will be ruled out in order not to unnecessarily obscure subject matters of the present invention.

Reference will now be made in detail to the embodiments of the present disclosure, examples of which are illustrated in the accompanying drawings.

It will be understood that, although the terms first, second, etc. may be used herein to describe various elements, these elements should not be limited by these terms. These terms are only used to distinguish one element from another.

Hereinafter, exemplary embodiments of the present invention will be described in detail with reference to the accompanying drawings.

The present invention discloses a method and apparatus for performing high-speed scanning using a pulse of relatively high light strength as excitation light.

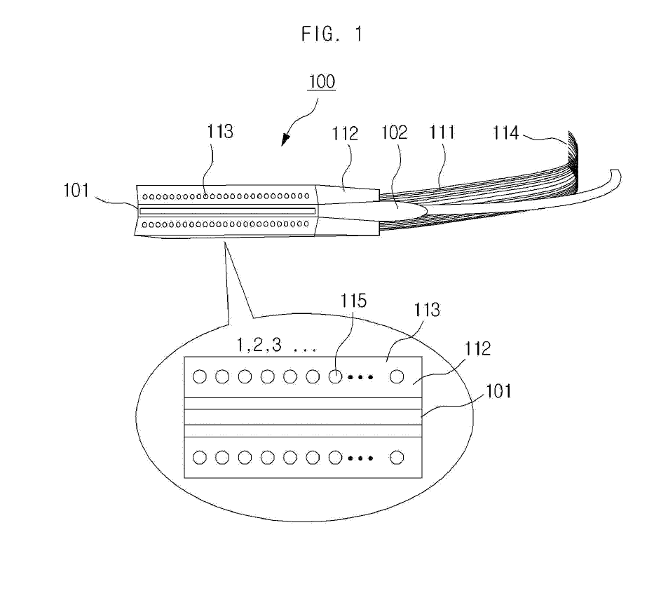

FIG. 1 is a block diagram showing an integrated photoacoustic probe that receives a laser photoexcitation and an ultrasonic wave for a photoacoustic image according to an embodiment of the present invention.

A photoacoustic probe 100 according to an embodiment of the present invention includes at least two optical fiber matrix connection modules 112 having a plurality of optical fiber strands 111 focused and connected thereto, an excitation laser output surface 113 formed on one end surface toward a biological tissue from the photoacoustic probe 100 and having a plurality of laser output holes 115 connected to the plurality of optical fiber strands 111 through a waveguide, and an ultrasonic wave sensor matrix 101 receiving an ultrasonic wave generated at the biological tissue due to laser light radiated through the excitation laser output surface 113.

Referring to FIG. 1, the optical fiber matrix connection modules 112 are formed on top and bottom surfaces of the photoacoustic probe 100 and configured to collectively connect the plurality of optical fiber strands 111 to the laser output holes 115 through an internal waveguide or an extended optical fiber such that the laser light may be sequentially output from the plurality of optical fiber strands 111.

According to an embodiment of the present invention, the photoacoustic probe 100 has one surface for transmitting laser light and receiving an ultrasonic wave generated inside a skin in response to the laser light. Excitation laser light is input through an input stage 114, which is the other end of the optical fiber strands 111. The optical fiber matrix connection modules 112 are configured to arrange the optical fiber strands 111 in a certain direction such that the input excitation laser light is guided from the inside and radiated closely when the optical fiber matrix connection modules 112 are brought in contact with the biological tissue.

According to an embodiment of the present invention, the laser light is output to the laser output holes 115 formed on the excitation laser output surface 113 through the optical fiber strands 111 that are fixed to the optical fiber matrix connection module 112 to be radiated to the biological tissue from which a tomographic image is to be obtained.

In other words, the photoacoustic 100 probe comprising, to performs transmitting laser light pulse and receiving an ultrasonic wave generated inside a skin in response to the laser light pulse at one surface

Further, the optical fiber matrix connection modules 112 may be disposed proximate to the ultrasonic wave sensor matrix 101 and disposed linearly or in a predetermined pattern.

Further, the optical fiber matrix connection module 112 may be disposed in various patterns such that the excitation laser light may reach the deepest region of the biological tissue.

The excitation laser output holes 115 are arranged to guide the excitation laser light from the optical fiber strands 111 inside the optical fiber matrix connection module 112.

In addition, the excitation laser output hole 115 may be a surface of an optical focusing component that focuses the excitation laser light at a predetermined depth, such as a graded-index (GRIN) lens.

According to an embodiment of the present invention, the excitation laser output surface 113 is formed at top and bottom sides of one end surface of the photoacoustic probe 100, and the ultrasonic wave sensor matrix 101 is formed in a middle side of the end surface of the photoacoustic probe 100.

The ultrasonic wave sensor matrix 101 collects an ultrasonic wave generated inside a skin in response to laser light radiated by the excitation laser output surface 113 and transmits the collected ultrasonic wave to a control unit as an electrical signal.

The ultrasonic wave sensor matrix 101 may include an ultrasonic focusing acoustic lens such that an ultrasonic wave may be effectively detected at a certain predetermined depth.

The ultrasonic focusing acoustic lens may be formed of a material such as silicone rubber having an acoustic impedance similar to that of the examined body. Furthermore, the ultrasonic focusing acoustic lens may perform focusing in a convex form.

A plurality of ultrasonic wave sensors are disposed on the ultrasonic wave sensor matrix 101.

An ultrasonic wave sensor matrix module 102 may be configured to input an ultrasonic wave to each ultrasonic wave sensor in the ultrasonic wave sensor matrix 101 having the ultrasonic wave sensors that are electrically separated and include an ultrasonic wave absorber structure to prevent the input ultrasonic wave from being reflected.

The ultrasonic wave absorber structure may be formed of an attenuation material having a low acoustic impedance.

The collected detected ultrasonic signal may be output as an electrical signal through the ultrasonic wave sensor matrix 101. A two-dimensional tomographic image may be obtained through combination and analysis of the collected signal.

Each detected signal may be electrically amplified, beam-formed around any position toward the ultrasonic wave sensor matrix 101, and focused at a specific depth of the biological tissue.

According to an embodiment of the present invention, one end of several optical fiber strands is used as an output hole of an excitation laser beam for a photoacoustic image, and the other end is used for injecting and scanning the excitation light.

When sequential laser beam pulses are radiated to a skin, specific ultrasonic waves are generated while an internal tissue such as blood vessel is inflated or deflated due to the laser beam pulses. A two-dimensional image indicating a characteristic structure under the skin may be obtained by combining and imaging the specific ultrasonic signal matrix.

That is, an ultrasonic signal output in response to a laser beam transmitted from one optical fiber strand is an A-scan signal obtained in a depth direction of one point of an object of a biological tissue, and a B-scan image obtained by sequentially combining position points of the A-scan signals is a two-dimensional image for recognizing a characteristic structure (an inflammatory area or cancerous area different from other tissue) under the skin.

According to an embodiment of the present invention, the other end for injecting and scanning the excitation light is formed in the form of a circle that is a disposition structure of the optical connection lens.

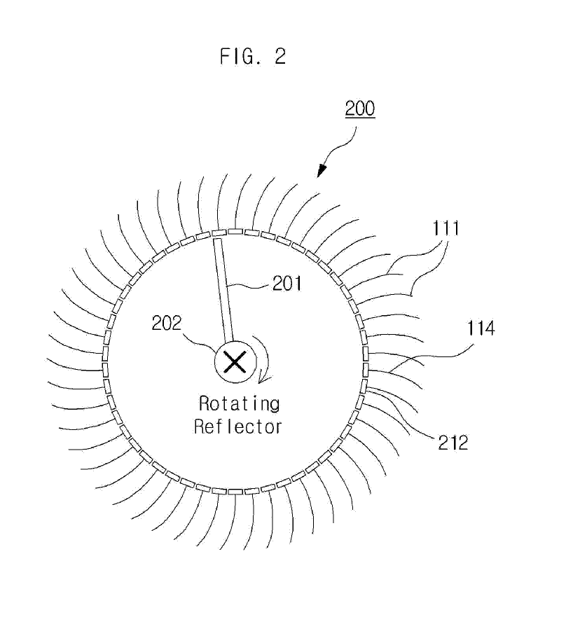

FIG. 2 shows a structure of a laser delivery device that outputs an excitation laser beam according to an embodiment of the present invention.

A laser delivery device 200 is configured to receive an excitation laser beam generated by an excitation laser beam generator and guide laser light pulses having sequential time intervals for optical scanning to input stages of the optical fiber strands.

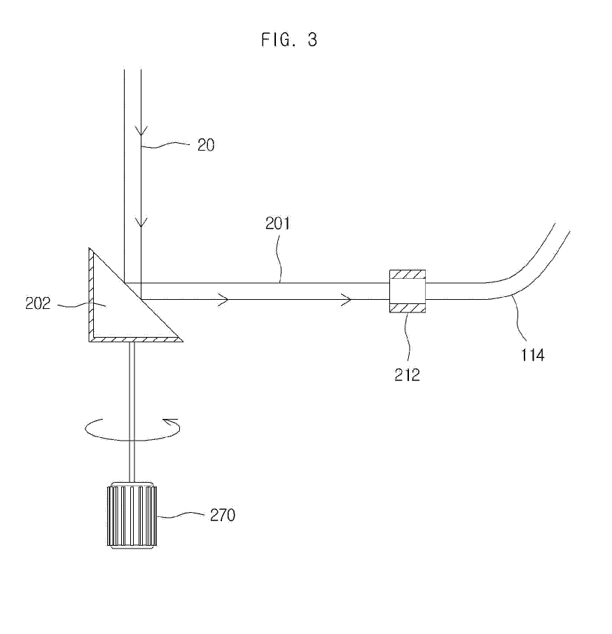

The laser delivery device 200 according to an embodiment of the present invention includes a rotation reflector 202 that receives the excitation laser beam generated by the laser beam generator and reflects an excitation laser beam 20 at an angle, a plurality of optical connection lenses 212 disposed to form a circle having a predetermined radius from the rotation reflector 202, and a driving device 270 in FIG. 3 that rotates the rotation reflector 202.

FIG. 3 is a view for illustrating a traveling path of a laser beam reflected by the rotation reflector 202 of a laser scanning device according to an embodiment of the present invention.

According to an embodiment of the present invention, the rotation reflector 202 is configured to reflect an excitation laser beam 20, which is emitted by the laser beam generator at an angle of 45 or 135 degrees with respect to the excitation laser beam 20 generated from the laser beam generator, at an angle of 90 degrees to optically and sequentially connect the excitation laser beam to the optical connection lenses 212 having a certain distance through rotation.

The angle is merely intended to illustrate an embodiment. In actual manufacturing, the rotation reflector 202 may be formed at various angles with respect to a laser beam and rotated to reflect the excitation laser beam 20, which is emitted by the laser beam generator, at various angles to optically connect the reflected excitation laser beam 20 to a plurality of optical fiber strands through the plurality of optical connection lenses 212.

The laser beam 20 that is incident on the rotating mirror Is divided into a laser light pulse 201 by the rotation of the reflecting mirror 202.

In other words, the rotation reflector 202 deliver laser light pulses 201 separated radially through rotation to the circumference side of rotation radius.

The excitation laser beam 20 for a photoacoustic image is incident in the same axial direction as a rotation axis of the rotation reflector 202 for performing reflection and optical connection.

The excitation laser beam 20 incident to the rotation reflector 202 is reflected by the rotation reflector 202. Thus, laser light pulses 201 are radially and sequentially connected to the plurality of optical connection lenses 212.

Each of the plurality of optical connection lenses 212 is connected with the input stage 114 of each optical fiber strand. The excitation laser light pulses 201 incident to the input stage 114 of each of the plurality of optical fiber strands is guided to the optical fiber strand 111 through the input stage 114 and radiated by the photoacoustic probe 100 onto an object of a biological tissue.

The laser delivery device 200 includes a plurality of optical connection lenses 212 disposed on a circumference while maintaining a predetermined radius from the rotation reflector 202. The plurality of optical connection lenses 212 are arranged such that the laser light pulses 201 is connected to the optical fiber strand 111. That is, one optical fiber strand 111 may be connected with one optical connection lens 212.

According to an embodiment of the present invention, each optical connection lens 212 may have a focusing lens disposed at the center, a lens base disposed and configured to fix the focusing lens at an edge thereof, and an optical fiber strand coupled to a back side of the focusing lens.

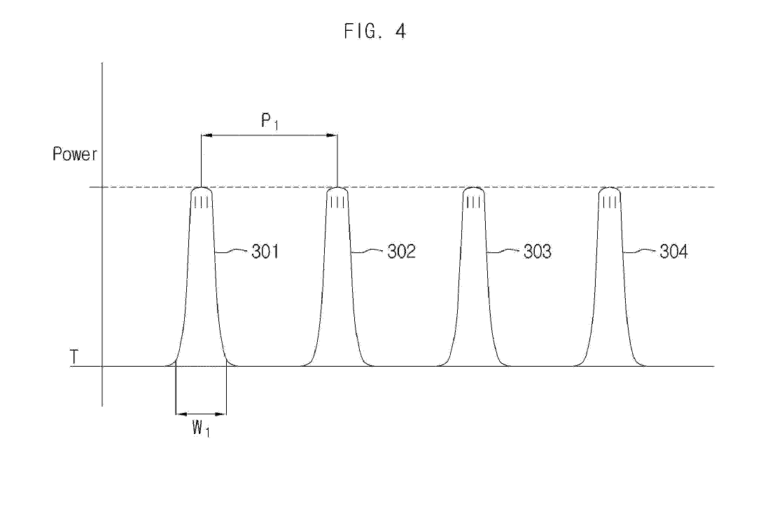

FIG. 4 shows a plurality of laser light pulses 201 input to a laser delivery device or output from a laser scanning apparatus according to an embodiment of the present invention.

According to an embodiment of the present invention, the rotation of the rotation reflector 202 may be set such that a laser beam pulse is incident when the reflected laser light pulses 201 is directed to a center of the optical connection lens 212 or set to be repeatedly stopped and rotated.

Referring to FIG. 4, since a width w1 of a pulse of the laser beam incident to the rotation reflector 202 is short (that is, several to tens of nanoseconds), and a period p1 between pulses is wide (that is, hundreds of microseconds to several milliseconds), the pulses of the laser beam reflected by the rotation reflector 202 may be controlled to be radiated to a center of the optical connection lens 212 only by simply adjusting a rotation phase. That is, the laser pulse input to the laser delivery device has the same temporal characteristic of the laser pulse as the laser pulse output from the laser delivery device. However, the laser delivery device performs spatial scanning when the laser pulse is delivered to the photoacoustic probe.

The rotation reflector 202 may be coupled with the driving device 270 such as a high-speed rotating motor. While the rotation reflector 202 rotates, the laser light pulses 201 may be sequentially radiated to the plurality of optical fiber strands 111 through the plurality of optical connection lenses 212, and thus guided to the photoacoustic probe 100.

One or more laser light pulses 201 input through the optical fiber strands 111 are radiated onto an object of a biological tissue through the laser output hole 115 according to a certain optical fiber matrix arrangement of the optical fiber matrix connection modules 112 of the photoacoustic probe 100.

Each laser light pulses 201 input through the optical fiber strand 111 is radiated and scanned onto an object of a biological tissue through the laser output hole 115 according to a certain optical fiber matrix arrangement of the optical fiber matrix connection modules 112 of the photoacoustic probe 100.

A method of scanning excitation light for a photoacoustic image according to an embodiment of the present invention includes generating an excitation laser beam from a laser beam generator; radially dividing and reflecting the generated excitation laser beam using a rotation reflector; sequentially inputting laser light pulses reflected by the rotation reflector to a plurality of optical connection lenses disposed in a circular form having a predetermined radius from the rotation reflector; guiding the laser light pulses input to the plurality of optical connection lenses to a photoacoustic probe through a plurality of optical fiber strands that are connected with the plurality of optical connection lenses respectively, radiating the laser light pulses guided to the photoacoustic probe onto a targeted tissue through a laser output hole; and receiving an ultrasonic wave generated at the targeted tissue in response to the radiated laser light pulses using an ultrasonic wave sensor matrix of the photoacoustic probe.

According to an embodiment of the present invention, a rotation phase of the rotation reflector 202 may be controlled by a control unit (not shown) such that the laser light pulse 201 is smoothly optically connected and synchronized when the excitation laser light pulses 201 is positioned at a center of each optical connection lens 212.

Alternatively, synchronization of optical connection may be easily performed by rotating all positions of the optical connection lenses 212 by a predetermined angle.

According to an embodiment of the present invention, a photoacoustic signal may be obtained and imaged with relatively low pulse energy, by radiating laser light pulses having peak outputs of the same level as the peak values of the excitation laser beam that are sequentially generated by the excitation laser beam generator through a mechanical structure of the excitation laser output surface 113 formed on the photoacoustic probe 100.

According to an embodiment of the present invention, an output generated by the laser apparatus may be efficiently delivered to the laser output surface of the photoacoustic probe directly without division by sequentially radiating the excitation laser light pulses to appropriate areas through the excitation laser output surface 113 formed on the photoacoustic probe 100.

In general, a laser beam output by one laser generator is divided into n beams in order to sequentially radiate a laser light onto n sections of a wide area. In this case, the output generated from one laser beam is reduced to 1/n, thus requiring the laser generator to have n times the output needed by a laser output surface.

According to an embodiment of the present invention, when the laser beam output by one laser generator is divided into n beams temporally and then transmitted through optical fiber strands, power outputs of the laser beams at output surfaces of all the optical fiber strands may be delivered to the laser output surface of the photoacoustic probe directly without reducing pulse power, thus radiating the laser light pulses to wide area using a laser beam having a relatively small output, compared to an existing laser beam generator.

That is, the existing technology in which an excitation laser beam is divided and radiated in order to irradiate a wide area requires an output amplified by the number of division. However, an apparatus for scanning excitation laser according to an embodiment of the present invention have a positive economic effect in that a laser light pulses having a peak output having the same level as a peak output of the excitation laser beam generated by the excitation beam generator may be uniformly radiated.

Furthermore, considering a position in which an excitation laser beam is radiated when acquiring a photoacoustic tomographic image of a targeted biological tissue, the accurate beam-forming may be easily performed by controlling a laser generation output and a rotation speed with respect to any position in a direction of the ultrasonic wave sensor matrix 101 in a relatively short time.

According to an embodiment of the present invention, the laser output surface 113 may be disposed on one end surface of the photoacoustic probe linearly or in a predetermined pattern, and the optical fiber matrix connection module 112 may be disposed on one end surface of the photoacoustic probe linearly or in a predetermined pattern.

According to an embodiment of the present invention, there is provided an apparatus for scanning excitation light that may radiate a high-energy pulse laser beam generated by a laser generator to a wide area suitable for a measured object in a form of sequentially-delayed laser light, without dividing an output of the high-energy pulse laser beam, thus delivering the entire output to a targeted biological tissue and allowing a high speed scanning

Furthermore, considering a position in which an excitation laser light pulses is radiated when acquiring a photoacoustic tomographic image of a targeted biological tissue, accurate beam-forming may be easily performed based on a position in a direction of an ultrasonic wave sensor matrix in a relatively short time.

The laser light pulses may be focused and scanned using a mechanical structure that rotates at a high speed according to an embodiment of the present invention, thus providing the method and apparatus for scanning excitation light, which are implemented at low costs.

According to an embodiment of the present invention, a photoacoustic signal may be obtained and imaged with relatively low pulse energy.

According to an embodiment of the present invention, a high-speed scanning may be enabled, thus obtaining a photoacoustic tomographic image in real time.

In addition, the present invention may be applied to a photoacoustic tomographic image.

The invention has been described with reference to preferred embodiment. It will be apparent to those skilled in the art that various modifications can be made to the above-described exemplary embodiments of the present invention without departing from the spirit or scope of the invention.

What is claimed is:

1. An apparatus for scanning excitation light for a photoacoustic image, which includes a laser delivery device, the laser delivery device comprising:
a rotation reflector configured to receive an excitation laser beam emitted from a laser beam generator, reflect the received excitation laser beam at a certain angle, and deliver laser light pulses separated radially through rotation toward a circumference of rotation radius;
a plurality of optical connection lenses disposed on a circumference having a predetermined radius from the rotation reflector and receiving the laser light pulses delivered by the rotation reflector sequentially at an interval; and
a plurality of optical fiber strands connected with the plurality of optical connection lenses, respectively, and configured to guide the laser light pulses incident at the optical connection lenses to a photoacoustic probe.

2. The apparatus of claim 1, further comprising the photoacoustic probe connected with the laser delivery device, and wherein the photoacoustic probe is configured to transmit the laser light pulses and receive an ultrasonic wave generated inside a skin in response to the laser light pulses on a same surface.

3. The apparatus of claim 2, wherein the photoacoustic probe comprises: at least two optical fiber matrix connection modules having the plurality of optical fiber strands collectively connected thereto;
an excitation laser output surface having a plurality of laser output holes formed therein, the plurality of laser output holes being connected with the plurality of optical fiber strands coupled through the optical fiber matrix connection modules and configured to output the laser light pulses; and an ultrasonic wave sensor matrix module disposed adjacent to the laser output surface and configured to receive an ultrasonic wave generated at a targeted tissue by the laser light pulses radiated to the targeted tissue.

4. The apparatus of claim 1, wherein the rotation reflector is coupled with a driving device rotating at a high speed, and wherein the laser light pulses are input to the plurality of optical connection lenses while the rotation reflector is rotated.

5. The apparatus of claim 1, wherein the rotation reflector is disposed at an angle of 45 degrees or 135 degrees with respect to the excitation laser beam to reflect the excitation laser beam by 90 degrees.

6. The apparatus of claim 3, wherein the excitation laser output surface is formed at top and bottom sides of one end surface of the photoacoustic probe, and the ultrasonic wave sensor matrix is formed at a middle side of the end surface of the photoacoustic probe, and
the optical fiber matrix connection modules are formed on top and bottom surfaces of the photoacoustic probe.

7. The apparatus of claim 3, wherein the excitation laser output surface is disposed on one end surface of the photoacoustic probe either linearly or in a predetermined pattern, and
the optical fiber matrix connection modules are disposed on one end surface of the photoacoustic probe either linearly or in a predetermined pattern.

8. The apparatus of claim 3, wherein the ultrasonic wave sensor matrix module further comprises an ultrasonic wave absorber structure configured to prevent an input ultrasonic signal from being reflected.

9. The apparatus of claim 3, wherein the ultrasonic wave sensor matrix module further comprises an ultrasonic wave focusing acoustic lens to effectively detect an ultrasonic wave at a certain predetermined depth of an examined object.

10. A method of scanning excitation light for a photoacoustic image, the method comprising:
generating an excitation laser beam from a laser beam generator;
reflecting the generated excitation laser beam at a predetermined angle by a rotation reflector and sequentially delivering laser light pulses toward a circumference of a rotation radius;
inputting the laser light pulses to a plurality of optical connection lenses, the plurality of optical connection lenses being disposed in a circular form having a predetermined radius from the rotation reflector;
guiding the laser light pulses input to the plurality of optical connection lenses to a photoacoustic probe through a plurality of optical fiber strands connected to the plurality of optical connection lenses, respectively;
radiating the laser light pulses guided to the photoacoustic probe onto a targeted tissue through a plurality of laser output holes; and
receiving an ultrasonic wave generated at the targeted tissue by the radiated laser light pulses using an ultrasonic wave sensor matrix of the photoacoustic probe.

11. The method of claim 10, wherein the rotation reflector is coupled with a driving device rotating at a high speed, and wherein the laser light pulses having the excitation laser beam sequentially divided therein are input to the plurality of optical connection lenses, respectively, as the rotation reflector is rotated, and a rotation phase of the driving device is controlled by a control device such that the laser light pulses are input when the laser light pulses are positioned at a center of each of the optical connection lenses.

12. The method of claim 10, wherein the rotation reflector is disposed at an angle of 45 degrees or 135 degrees with respect to the excitation laser beam to reflect the excitation laser beam by 90 degrees.
